(19) United States
(12) Patent Application Publication
(10) Pub. No.: US 2009/0299264 A1
(43) Pub. Date: Dec. 3, 2009

(54) Electrode Assembly for Dry Type Iontophoresis

(75) Inventors: Takehiko Matsumura, Tokyo (JP); Mizuo Nakayama, Tokyo (JP); Hidero Akiyama, Tokyo (JP); Akihiko Matsumura, Tokyo (JP)

Correspondence Address: SEED INTELLECTUAL PROPERTY LAW GROUP PLLC, 701 FIFTH AVE, SUITE 5400, SEATTLE, WA, 98104, US

(73) Assignee: TTI ELLEBEAU, INC., Tokyo (JP)

(21) Appl. No.: 11/992,671

(22) PCT Filed: Sep. 28, 2006
(86) PCT No.: PCT/JP2006/319295

§ 371 Date: May 8, 2009

(30) Foreign Application Priority Data
Sep. 28, 2005 (JP) 2005-281908

(51) Int. Cl. A61N 1/30

(52) U.S. Cl. 604/20

(57) ABSTRACT

An iontophoresis device capable of stably holding a drug for a long time period and of transdermally administering the drug at a high transport number during use. The iontophoresis device includes a drug holding portion to store the ionic drug in a dry state and at least an aqueous medium holding portion to store an aqueous medium. The drug holding portion exhibiting ion conductivity in response to a needle inserted into the aqueous medium holding portion from outside the electrode assembly such that the drug holding portion is supplied with the aqueous medium during use and the ionic drug dissolves in the aqueous medium.

### TECHNICAL FIELD

The present invention relates to a technique of transdermally administering various ionic drugs (transdermal drug delivery) by iontophoresis. In particular, the present invention relates to an electrode assembly for iontophoresis capable of stably holding a drug for a long time period and of transdermally administering the drug at a high transport number during use.

### BACKGROUND ART

A method of introducing (permeating) an ionic drug placed on the surface of the skin or mucosa (hereinafter, merely referred to as “skin”) of a predetermined site of an organism into the body through the skin by giving the skin an electromotive force sufficient to drive such an ionic drug is called iontophoresis (iontophorese, ion introduction method, ion permeation therapy) (see, for example, Patent Document 1).

For example, positively charged ions are driven (transported) into the skin on the side of an anode (positive electrode) in an electric system of an iontophoresis device. On the other hand, negatively charged ions are driven (transported) into the skin on the side of a cathode (negative electrode) in the electric system of the iontophoresis device.

Conventionally, a large number of such iontophoresis devices as described above have been proposed (see, for example, Patent Documents 1 to 7).

Such conventional iontophoresis device as described above is requested to administer a drug to an organism at a high transport number (transport efficiency) during use in order to secure a sufficient therapeutic effect and to stably hold the drug during a storage period. However, the drug held by the iontophoresis device in advance may be lost owing to, for example, an irreversible change such as the leakage, hydrolysis, association, or aggregation of the drug depending on, for example, the duration of the storage period and the kind of the drug. When an ionic additive is applied in order to maintain the stability of an ionic drug, such ionic additive may compete with the drug to reduce the transport number of the drug into an organism during use. Therefore, it is important for an iontophoresis device to stably hold a drug and to secure a high transport number of the drug during use.
[Patent Document 1] JP 63-35266 A
[Patent Document 2] JP 04-297277 A
[Patent Document 3] JP 2000-229128 A
[Patent Document 4] JP 2000-229129 A
[Patent Document 5] JP 2000-237327 A
[Patent Document 6] JP 2000-237328 A
[Patent Document 7] WO 03/037425 A1

### DISCLOSURE OF THE INVENTION

### Problems to be Solved by the Invention

The present invention has been made in view of the above-described problems of the prior art, and an object of the present invention is to provide an electrode assembly for iontophoresis capable of stably holding a drug before use and of transferring the drug into an organism at a high transport number during use and an iontophoresis device including the electrode assembly for iontophoresis.

### Means for Solving the Problems

To solve the above problems, according to one aspect of the present invention, there is provided an electrode assembly for iontophoresis for administering an ionic drug to an organism transdermally by releasing the ionic drug by iontophoresis, characterized by including at least a constituent material that exhibits conductivity by being supplied with an aqueous medium, the constituent material holding the ionic drug in a dry state, the aqueous medium being supplied to the constituent material during use to dissolve the ionic drug in the aqueous medium.

According to a preferred aspect of the present invention, the electrode assembly for iontophoresis is characterized by including: an electrode connected to an electric power source with the same polarity as that of a drug component of the ionic drug; an electrolyte solution holding portion holding an electrolyte solution, the electrolyte solution holding portion being placed adjacent to the electrode; a first ion exchange membrane selecting an ion having a polarity opposite to that of a charged ion of the ionic drug, the first ion exchange membrane being placed adjacent to the electrolyte solution holding portion; a drug holding portion holding the ionic drug, the drug holding portion being placed adjacent to the first ion exchange membrane; and a second ion exchange membrane selecting an ion having the same polarity as that of the charged ion of the ionic drug, the second ion exchange membrane being placed adjacent to the drug holding portion, at least the drug holding portion being in a dry state, the aqueous medium being supplied to the drug holding portion during use of the electrode assembly for iontophoresis to dissolve the ionic drug in the aqueous medium.

According to another aspect of the present invention, there is provided an iontophoresis device characterized by including the electrode assembly.

### Effect of the Invention

As described above, each of the electrode assembly for iontophoresis and the iontophoresis device including the same according to the present invention is capable of stably holding an ionic drug for a long time period before use and of transferring the drug into an organism at a high transport number during use. In addition, the electrode assembly eliminates the need for using an ionic additive for storing an ionic drug, so the ionic drug can be efficiently released during use.

### BEST MODE FOR CARRYING OUT THE INVENTION

Electrode Assembly for Iontophoresis

As described above, the electrode assembly for iontophoresis for administering an ionic drug to an organism transdermally by releasing the ionic drug by iontophoresis is characterized by including at least a constituent material that exhibits conductivity by being supplied with an aqueous medium, the constituent material holding the ionic drug in a dry state, the aqueous medium being supplied to the constituent material during use to dissolve the ionic drug in the aqueous medium.

Hereinafter, the present invention will be described with reference to preferred specific examples shown in the figures.

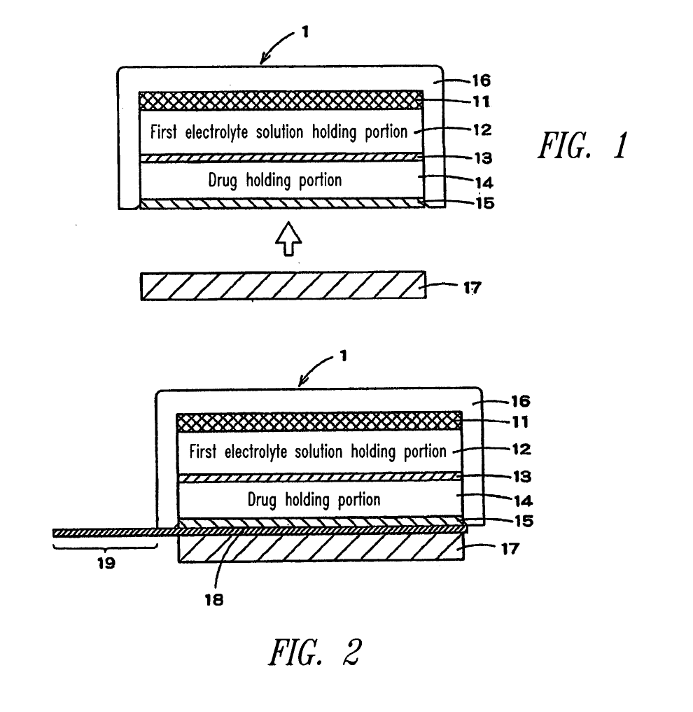

FIG. 1 shows an embodiment of an electrode assembly 1 for iontophoresis. The electrode assembly 1 for iontophoresis includes: an electrode 11 connected to an electric power source with the same polarity as that of a drug component of an ionic drug; an electrolyte solution holding portion 12 holding an electrolyte solution, the electrolyte solution holding portion 12 being placed adjacent to the electrode 11; a first ion exchange membrane 13 selecting an ion having a polarity opposite to that of a charged ion of the ionic drug, the first ion exchange membrane 13 being placed adjacent to the electrolyte solution holding portion 12; a drug holding portion 14 holding the ionic drug, the drug holding portion 14 being placed adjacent to the first ion exchange membrane 13; and a second ion exchange membrane 15 selecting an ion having the same polarity as that of the charged ion of the ionic drug, the second ion exchange membrane 15 being placed adjacent to the drug holding portion 14, and they are stored in a cover or container 16. In addition, the drug holding portion 14 is in a dry state. The term “holding portion” as a constituent member for the foregoing and the following includes a layer form and a membrane form. In addition, each of the electrolyte solution holding portion 12, the first ion exchange membrane 13, the drug holding portion 14, and the second ion exchange membrane 15 is placed in a dry state. A peelable sheet (not shown) may be stuck to the external surface of the second ion exchange membrane 15, and the sheet is peeled at the time of use.

The electrode assembly 1 for iontophoresis further includes an aqueous medium holding portion 17 holding an aqueous medium, the aqueous medium holding portion 17 being detachably placed. The aqueous medium is supplied from the aqueous medium holding portion 17 to the drug holding portion 14 during use of the electrode assembly 1 for iontophoresis to dissolve the ionic drug in the aqueous medium. In FIG. 1, the aqueous medium can be supplied to the drug holding portion 13 via the second ion exchange membrane 15 by bringing the aqueous medium holding portion 17 and the second ion exchange membrane 15 into contact with each other.

FIG. 2 shows another embodiment of the electrode assembly 1 for iontophoresis. The electrode assembly 1 for iontophoresis further includes a removable separator portion 18 placed adjacent to the second ion exchange membrane 15. The separator portion 18 separates the aqueous medium holding portion 17 and the second ion exchange medium 15 each placed adjacent to the separator portion 18 from each other.

The separator portion 18 may include an auxiliary grip 19 for removing the separator portion 18. During use of the electrode assembly 1 for iontophoresis, for example, the separator portion 18 is removed by being pulled so that the aqueous medium holding portion 17 and the second ion exchange membrane 15 are brought into contact with each other. As a result, the aqueous medium can be supplied to the drug holding portion 14 via the second ion exchange membrane 15.

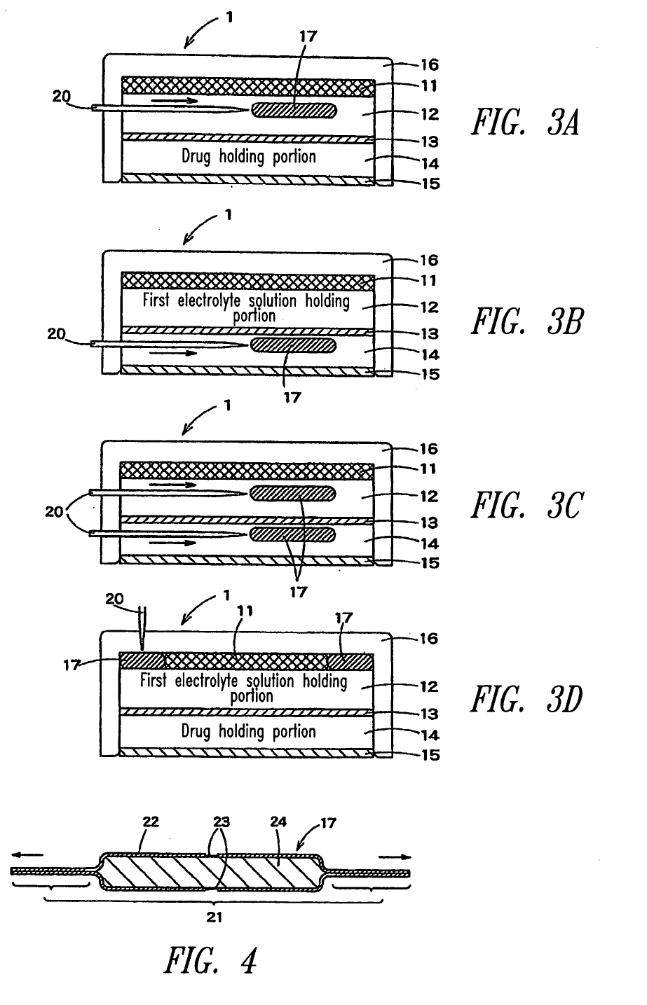

FIGS. 3 each show another embodiment of the electrode assembly for iontophoresis. In the electrode assembly 1 for iontophoresis, the aqueous medium holding portion 17 is formed as a bag-like sealing body having the aqueous medium sealed therein.

In FIG. 3(A), the aqueous medium holding portion 17 is placed in the electrolyte solution holding portion 12 in a dry state.

In FIG. 3(B), the aqueous medium holding portion 17 is placed in the drug holding portion 14 in a dry state.

In FIG. 3(C), the aqueous medium holding portion 17 is placed in the electrolyte solution holding portion 12 and the drug holding portion 14 each in a dry state.

In FIG. 3(D), the aqueous medium holding portion 17 is juxtaposed with the electrode 11 to be adjacent to the electrolyte solution holding portion 12 in a dry state. In FIG. 3(D), the aqueous medium holding portion 17 is juxtaposed so as to surround the electrode assembly 11 of the electrode 11. However, the placement of the aqueous medium holding portion 17 on the electrolyte solution holding portion 12 can be appropriately changed as long as energization during use is not inhibited.

In each of FIGS. 3(A), 3(B), 3(C), and 3(D), the bag-like aqueous medium holding portion 17 can be opened by inserting, for example, a needle 20 into the aqueous medium holding portion 17 from the outside of the electrode assembly 1 for iontophoresis during use as shown in each of FIGS. 3(A), 3(B), and 3(C). When the aqueous medium holding portion 17 is formed of a base material that can be molten by heat or electricity, the aqueous medium holding portion 17 can be opened by a current or heat upon energization.

In addition, as shown in FIG. 4, the aqueous medium holding portion 17 may include auxiliary grips 21 for opening. In such embodiment, a central portion 23 of the base material 22 for the aqueous medium holding portion 17 can be formed into a recess in consideration of convenience for opening. During use, the aqueous medium holding portion 17 is opened by pulling the grips 21 for opening from both sides, whereby an aqueous medium 24 can be released. The aqueous medium holding portion as a sealing body shown in FIG. 4 can be similarly used in the embodiment shown in FIG. 1.

In each of FIGS. 1, 2, 3, and 4, at least the drug holding portion is in a dry state. However, for stably holding a drug, each of the electrolyte solution holding portion, the drug holding portion, the first ion exchange membrane, and the second ion exchange membrane is preferably in a dry state. In addition, each of those portions can be dried by means of a conventionally known drying device such as a vacuum pump. For example, the drug holding portion can be dried by means of a vacuum pump after the constituent member of the drug holding portion is impregnated with an ionic drug and an aqueous medium.

In each of the embodiments shown in FIGS. 1 and 2, the aqueous medium holding portion can be formed by, for example, impregnating a non-woven fabric or a water-absorbing cross-linkable polymer with an aqueous medium. In particular, when the aqueous medium holding portion is placed so as to be detachable from the electrode assembly for iontophoresis as shown in FIG. 1, the aqueous medium holding portion can be a bottle containing a required amount of aqueous medium.

Furthermore, when the aqueous medium holding portion serves as a sealing body for an aqueous medium as shown in each of FIGS. 3 and 4, the aqueous medium can be sealed through heat sealing or the like by means of, for example, a thermoplastic resin as a base material. Any conventionally known approach can be employed as an approach to placing such aqueous medium holding portion in the electrolyte solution holding portion or the drug holding portion. For example, after an aqueous medium holding portion produced in advance as a sealing body has been placed in the constituent member of each of the electrolyte solution holding portion and the drug holding portion, each member may be subjected to compression molding.

When the aqueous medium holding portion is equipped with the grips for opening, these grips are appropriately arranged by one skilled in the art to project toward the outside of the electrode assembly.

Examples of the aqueous medium in the aqueous medium holding portion include water and an electrolyte solution to be described later. Of those, an electrolyte solution is preferable. The use of such electrolyte solution makes the electrode assembly according to the present invention quickly applicable to iontophoresis. In consideration of, for example, the nature of an ionic drug, a conventionally known nonionic additive such as a parahydroxybenzoate can be appropriately added to the aqueous medium.

Iontophoresis Device

In addition, as described above, the electrode assembly according to the present invention can be suitably used as a working electrode assembly in an iontophoresis device. Hereinafter, an iontophoresis device including the electrode assembly according to the present invention will be described with reference to preferred specific examples shown in the figures.

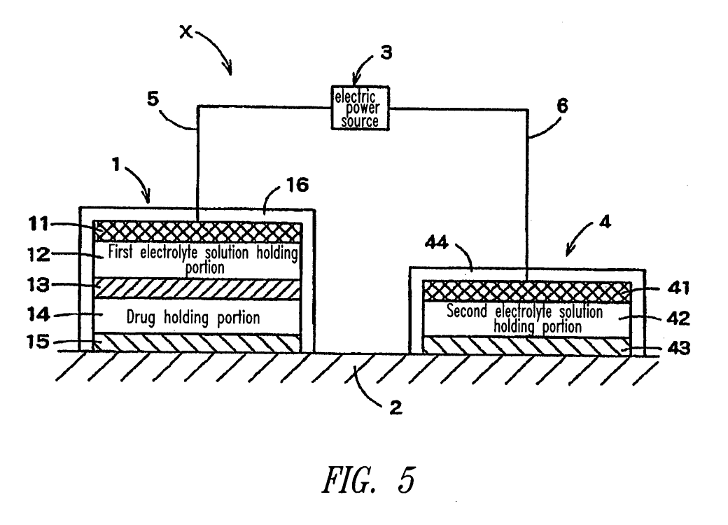

The embodiment shown in FIG. 5 shows a state where an iontophoresis device X including the electrode assembly according to the present invention shown in FIG. 1 as a working electrode assembly is placed on the surface of a skin 2. The iontophoresis device X further includes: an electric power source device 3; and a non-working electrode assembly 4 (a ground electrode assembly) as a counter electrode of the working electrode assembly 1. Here, an electrolyte solution as an aqueous medium has been already supplied to the working electrode assembly 1. In addition, the working electrode assembly 1 is constituted by: the electrode 11 connected via a cord 5 to the side of the electric power source device 3 having the same polarity as that of a charged ion of a drug; the electrolyte solution holding portion 12 holding an electrolyte solution by being impregnated with the electrolyte solution, the electrolyte solution holding portion 12 being placed adjacent to the electrode 12; the first ion exchange membrane 13 selecting an ion having a polarity opposite to that of a charged ion of an ionic drug, the first ion exchange membrane 13 being placed adjacent to the electrolyte solution holding portion 12; the drug holding portion 14 holding the ionic drug by being impregnated with the ionic drug, the drug holding portion 14 being placed adjacent to the first ion exchange membrane 13; and the second ion exchange membrane 15 selecting an ion having the same polarity as that of the charged ion of the ionic drug, the second ion exchange membrane 15 being placed adjacent to the drug holding portion 14.

Meanwhile, the non-working electrode assembly 4 is constituted by: an electrode 41 having a polarity opposite to that of the electrode 11 in the working electrode assembly 1, the electrode 41 being connected via a cord 6 to the electric power source device 3; an electrolyte solution holding portion 42 holding an electrolyte solution by being impregnated with the electrolyte solution, the electrolyte solution holding portion 42 being placed adjacent to the electrode 41; and a second ion exchange membrane 43 selecting an ion having a polarity opposite to that of the charged ion of the ionic drug, the second ion exchange membrane 43 being placed adjacent to the electrolyte solution holding portion 42, and they are stored in a cover or container 44.

In the iontophoresis device X, upon energization with the electric power source 3, the ionic drug migrates by virtue of an electric field, and is transdermally administered to an organism via the ion exchange membrane 15. In this case, an ion having a polarity opposite to that of the ionic drug is prevented from transferring from the side of the organism into the side of the drug holding portion 14 by the action of each of the ion exchange membranes 13 and 15. Thus, the movement of H+ or OH− generated on the electrode 11 to the side of the skin 21 is suppressed. Accordingly, the ionic drug can be stably and efficiently administered for a long time period while a change in pH on the skin 2 is suppressed.

In addition, the constituent material holding an ionic drug in a dry state according to the present invention preferably has a sufficient ability of holding the drug by being impregnated with the drug, and a sufficient ability (ion transferability, ion conductivity) of transferring the ionic drug impregnated into and held by the material to the skin side under predetermined electric field conditions. Examples of such constituent material include an acrylic hydrogel, a segmented polyurethane-based gel, and an acrylonitrile-based copolymer.

An example of the acrylic hydrogel includes a gel composed of 2-hydroxyethyl methacrylate and ethylene glycol dimethacrylate. In such gel, a monomer ratio between 2-hydroxyethyl methacrylate and ethylene glycol dimethacrylate is preferably 98 to 99.5:0.5 to 2. Examples of such acrylic hydrogel as described above include those available from SUN-CONTACT LENS Co., Ltd.

An example of the segmented polyurethane-based gel includes a polyurethane-based copolymer having a polyethylene glycol segment and a polypropylene glycol segment. Such polyurethane-based gel can be synthesized by means of monomers (polyethylene glycol and polypropylene glycol) as segments and diisocyanate.

Examples of the acrylonitrile-based copolymer include: a copolymer of acrylonitrile and an alkyl (meth)acrylate having 1 to 4 carbon atoms; an acrylonitrile/vinyl acetate copolymer; an acrylonitrile/styrene copolymer; and an acrylonitrile/vinylidene chloride copolymer. In such acrylonitrile-based copolymer, the content of an acrylonitrile monomer is preferably 50 mol % or more, or more preferably 70 to 98 mol % or more. In addition, the acrylonitrile-based copolymer preferably has a porosity of 20 to 80%.

In addition, such constituent material as described above is preferably used as the constituent material of the drug holding portion. The drug holding portion is constituted as a thin film that holds an ionic drug. Such thin film can be used as an ion-conductive porous sheet for forming a gel-like solid electrolyte disclosed in, for example, JP 11-273452 A as desired. When such drug holding portion as described above is impregnated with a drug, an impregnation rate (defined by 100×(W−D)/D [%] where D indicates a dry weight and W indicates a weight after impregnation) is preferably 30 to 40%.

Specific examples of an ionic drug applicable to the electrode assembly for iontophoresis and the device including the same include: anesthetic drugs (such as procaine hydrochloride and lidocaine hydrochloride); hemostatic drugs (such as tranexamic acid and ε-aminocaproic acid); antibiotics (such as a tetracycline-based preparation, a kanamycin-based preparation, and a gentamicin-based preparation); vitamin (such as vitamin B1, vitamin B2, and vitamin C); adrenal cortex hormones (such as a hydrocortisone-based water-soluble preparation, a dexamethasone-based water-soluble preparation, and a prednisolone-based water-soluble preparation); and antibiotics (such as a penicillin-based water-soluble preparation and a chloramphenicole-based water-soluble preparation).

An ionic drug amount is determined for each individual ionic drug in such a manner that a preset effective blood concentration can be obtained for an effective time period upon application of the drug to a patient, and is set by one skilled in the art in accordance with, for example, the size and thickness of a drug holding portion or the like, the area of a drug release surface, a voltage in an electrode device, and an administration time.

In addition, an inactive electrode made of a conductive material such as carbon or platinum can be preferably used as the electrode of the electrode assembly.

The electrolyte solution holding portion can be constituted by a thin film capable of holding an electrolyte solution by being impregnated with the electrolyte solution. The thin film can be made of the same material as that used for the above-described drug holding portion.

A desired one can be appropriately used as the electrolyte solution depending upon the conditions such as a drug to be applied. However, an electrolyte solution that damages the skin of an organism owing to an electrode reaction should be avoided. An organic acid or a salt thereof present in a metabolic cycle of an organism is preferable as the electrolyte solution in the present invention in terms of harmlessness. For example, lactic acid and fumaric acid are preferable. Specifically, an aqueous solution of 1M of lactic acid and 1M of sodium fumarate (1:1) is preferable. Such electrolyte solution is preferable because: it has high solubility with respect to water and passes a current well; and in the case where a current is allowed to flow at a constant level, the electric resistance is low and a change in pH is relatively small in an electric power source device.

A cation exchange membrane and an anion exchange membrane are preferably used together as the first and second ion exchange membranes to be used for an electrode assembly. Preferable examples of the cation exchange membrane include NEOSEPTAs (CM-1, CM-2, CMX, CMS, CMB, and CLE04-2) manufactured by Tokuyama Co., Ltd. Preferable examples of the anion exchange membrane include NEOSEPTAs (AM-1, AM-3, AMX, AHA, ACH, ACS, ALE04-2, and AIP-21) manufactured by Tokuyama Co., Ltd. Other preferable examples include: a cation exchange membrane that includes a porous film having cavities a part or whole of which are filled with an ion exchange resin having a cation exchange function; and an anion exchange membrane that includes a porous film having cavities a part or whole of which are filled with an ion exchange resin having an anion exchange function.

The above-mentioned ion exchange resins can be fluorine-based ones that include a perfluorocarbon skeleton having an ion exchange group and hydrocarbon-based ones that include a nonfluorinated resin as a skeleton. From the viewpoint of convenience of production process, hydrocarbon-based ion exchange resins are preferably used. The filling rate of the porous film with the ion exchange resin, which varies depending on the porosity of the porous film, can be, for example, 5 to 95 mass %, and is preferably 10 to 90 mass %, or more preferably 20 to 60 mass %.

The ion exchange group in the above-mentioned ion exchange resin is not particularly limited so far as it is a functional group that generates a group having negative or positive charge in aqueous solutions. Such functional group may be present in the form of a free acid or a salt. Examples of a cation exchange group include a sulfonic group, a carboxylic acid group, and a phosphonic acid group. Of those, a sulfonic group is preferable. Examples of a counter cation for the cation exchange group include: alkali cations such as a sodium ion and a potassium ion; and ammonium ions. Examples of an anion exchange group include a primary amino group, a secondary amino group, a tertiary amino group, a quaternary ammonium group, a pyridyl group, an imidazole group, a quaternary pyridium group, and a quaternary imidazolium group. Of those, a quaternary ammonium group or a quaternary pyridium group is preferable. Examples of a counter cation for the anion exchange group include: halogen ions such as a chlorine ion; and hydroxy ions.

The above-mentioned porous film is not particularly limited and any porous film can be used as far as it is in the form of a film or sheet that has a large number of pores communicating both sides thereof. To satisfy both of high strength and flexibility, it is preferable that the porous film be made of a thermoplastic resin. Examples of the thermoplastic resin constituting the porous film include: polyolefin resins such as homopolymers or copolymers of α-olefins such as ethylene, propylene, 1-butene, 1-pentene, 1-hexene, 3-methyl-1-butene, 4-methyl-1pentene, and 5-methyl-1-heptene; vinyl chloride-based resins such as polyvinyl chloride, vinyl chloride-vinyl acetate copolymers, vinyl chloride-vinylidene chloride copolymers, and vinyl chloride-olefin copolymers; fluorine-based resins such as polytetrafluoroethylene, polychlorotrifluoroethylene, polyvinylidene fluoride, tetrafluoroethylene-hexafluoropropylene copolymers, tetrafluoroethylene-perfluoroalkyl vinylether copolymers, and tetrafluoroethylene-ethylene copolymers; polyamide resins such as nylon 66; and polyimide resins. Of those, polyolefin resins are preferable in consideration of, for example, mechanical strength, flexibility, chemical stability, and chemical resistance. Of those, polyethylene or polypropylene is more preferable, and polyethylene is still more preferable.

The properties of the above-mentioned porous film made of the thermoplastic resin are not particularly limited. However, the mean pore size is preferably 0.005 to 5.0 μm, more preferably 0.01 to 2.0 μm, or still more preferably 0.02 to 0.2 μm in consideration of the formation of an ion exchange membrane that is thin and has excellent strength and low electric resistance. The above-mentioned mean pore size as used herein means a mean flow pore size measured in conformance with the bubble point method (JIS-K3832-1990). Similarly, the porosity of the porous film is preferably 20 to 95%, more preferably 30 to 90%, or still more preferably 30 to 60%. In consideration of the thickness of an ion exchange membrane to be finally formed, the thickness of the porous film is preferably 5 to 140 μm, more preferably 10 to 130 μm, or still more preferably 15 to 55 μm. Usually, an anion exchange membrane or a cation exchange membrane formed of such porous film generally has the same thickness as that of the porous film or up to about 20 μm larger than the thickness of the porous film.

In addition, the following conditions are adopted as preferred energizing conditions in such iontophoresis device as described above.
(1) Constant current condition, specifically, 0.01 to 0.7 mA/cm², preferably 0.1 to 0.5 mA/cm²
(2) Safe voltage condition that realizes the above constant current, specifically, 50 V or less, preferably 30 V or less

Patent Document 7 according to the applicant of the present invention describes details about the above-described respective constituent materials, and the contents described in the document are also included in the present invention.

### BRIEF DESCRIPTION OF THE DRAWINGS

[FIG. 1] FIG. 1 is a schematic view of an electrode assembly according to the present invention including an aqueous medium holding portion that is detachably placed.

[FIG. 2] FIG. 2 is a schematic view of the electrode assembly according to the present invention including a separator portion.

[FIGS. 3] FIG. 3(A) is a schematic view of the electrode assembly according to the present invention in which the aqueous medium holding portion is placed in an electrolyte solution holding portion. FIG. 3(B) is a schematic view of the electrode assembly according to the present invention having placed therein the aqueous medium holding portion placed in a drug holding portion. FIG. 3(C) is a schematic view of the electrode assembly according to the present invention in which the aqueous medium holding portion is placed in the electrolyte solution holding portion and the drug holding portion. FIG. 3(D) is a schematic view of the electrode assembly according to the present invention in which the aqueous medium holding portion is juxtaposed with an electrode to be adjacent to the electrolyte solution holding portion.

[FIG. 4] FIG. 4 is a schematic view of the aqueous medium holding portion including a grip for opening and having an aqueous medium sealed therein.

[FIG. 5] FIG. 5 is a schematic view of an iontophoresis device including the electrode assembly according to the present invention.

### DESCRIPTION OF SYMBOLS

X IONTOPHORESIS DEVICE

1 ELECTRODE ASSEMBLY FOR IONTOPHORESIS (WORKING ELECTRODE ASSEMBLY)

11, 41 ELECTRODE

12, 42 ELECTROLYTE SOLUTION HOLDING PORTION

13 FIRST ION EXCHANGE MEMBRANE 13

14 DRUG HOLDING PORTION

15, 43 SECOND ION EXCHANGE MEMBRANE

16, 44 COVER OR CONTAINER

17 AQUEOUS MEDIUM HOLDING PORTION

18 SEPARATOR PORTION

19 AUXILIARY GRIP OF SEPARATOR PORTION

20 NEEDLE

21 AUXILIARY GRIP FOR OPENING AQUEOUS MEDIUM HOLDING PORTION

22 BASE MATERIAL FOR AQUEOUS MEDIUM HOLDING PORTION

23 CENTRAL PORTION OF AQUEOUS MEDIUM HOLDING PORTION

24 AQUEOUS MEDIUM

2 SKIN

3 ELECTRIC POWER SOURCE DEVICE

4 NON-WORKING ELECTRODE ASSEMBLY

5, 6 CORD

1. An electrode assembly to transdermally administer an ionic drug into an organism via iontophoresis, the electrode assembly comprising:
drug holding portion to store the ionic drug in a dry state before use; and
at least an aqueous medium holding portion to store an aqueous medium, the drug holding portion exhibiting ion conductivity in response to a needle inserted into the aqueous medium holding portion from outside the electrode assembly such that the drug holding portion is supplied with the aqueous medium during use and the ionic drug dissolves in the aqueous medium.

2. The electrode assembly of claim 1, further comprising:
an electric power source;
a first electrode electrically coupled to the electric power source to have a same polarity as a component of the ionic drug;
a first electrolyte solution holding portion impregnated with a first electrolyte solution, the first electrolyte solution holding portion disposed adjacent to the first electrode;
a first ion exchange membrane that substantially passes ions having a polarity that is the same as a polarity of the ionic drug and that substantially blocks ions having a polarity that is opposite the polarity of the ionic drug, the ion exchange membrane disposed adjacent to the first electrolyte solution holding portion, the drug holding portion impregnated with the ionic drug and disposed adjacent to the first ion exchange membrane; and
a second ion exchange membrane that substantially passes ions having a polarity opposite the polarity of the ionic drug and that substantially blocks ions having a polarity that is the same as a polarity of the ionic drug, the second ion exchange membrane disposed adjacent to the drug holding portion.

3. The electrode assembly of claim 2 wherein at least one of the first electrolyte solution holding portion, the first ion exchange membrane, and the second ion exchange membrane is in a dry state.

4. (canceled)

5. The electrode assembly of claim 43 wherein the aqueous medium holding portion is removably placed in the electrode assembly.

6-10. (canceled)

11. The electrode assembly of claim 5 wherein the aqueous medium holding portion is placed in at least one of the first electrolyte solution holding portion and the drug holding portion.

12. The electrode assembly of claim 5 wherein the aqueous medium holding portion is juxtaposed with the first electrode and positioned adjacent to the first electrolyte solution holding portion.

13. (canceled)

14. The electrode assembly of claim 5 wherein the aqueous medium holding portion takes a form of a bag-like sealing body having the aqueous medium sealed therein.

15. An electrode assembly to transdermally administer an ionic drug to an organism via iontophoresis, the electrode assembly comprising:
a drug holding portion to store the ionic drug in a dry state; and
at least an aqueous medium holding portion to store an aqueous medium, the drug holding portion exhibiting ion conductivity in response to the aqueous medium holding portion being molten by heat or electricity such that the drug holding portion is supplied with the aqueous medium during use and the ionic drug dissolves in the aqueous medium.

16. The electrode assembly of claim 15, further comprising:
an electric power source;
a first electrode electrically coupled to the electric power source to have a same polarity as a component of the ionic drug;
a first electrolyte solution holding portion impregnated with a first electrolyte solution, the first electrolyte solution holding portion disposed adjacent to the first electrode;
a first ion exchange membrane that substantially passes ions having a polarity that is the same as a polarity of the ionic drug and that substantially blocks ions having a polarity that is opposite the polarity of the ionic drug, the ion exchange membrane disposed adjacent to the first electrolyte solution holding portion, the drug holding portion impregnated with the ionic drug and disposed adjacent to the first ion exchange membrane; and
a second ion exchange membrane that substantially passes ions having a polarity opposite the polarity of the ionic drug and that substantially blocks ions having a polarity that is the same as a polarity of the ionic drug, the second ion exchange membrane disposed adjacent to the drug holding portion.

17. The electrode assembly of claim 16 wherein at least one of the first electrolyte solution holding portion, the first ion exchange membrane, and the second ion exchange membrane is in a dry state.

18. The electrode assembly of claim 17 wherein the aqueous medium holding portion is removably placed in the electrode assembly.

19. The electrode assembly of claim 18 wherein the aqueous medium holding portion is placed in at least one of the first electrolyte solution holding portion and the drug holding portion.

20. The electrode assembly of claim 18 wherein the aqueous medium holding portion is juxtaposed with the first electrode and positioned adjacent to the first electrolyte solution holding portion.

21. The electrode assembly of claim 18 wherein the aqueous medium holding portion takes a form of a bag-like sealing body having the aqueous medium sealed therein.

22. An iontophoresis device comprising:
an electric power source;
a first electrode assembly electrically coupled to the electric power source to store and transdermally administer an ionic drug to an organism via iontophoresis, the first electrode assembly includes:
a drug holding portion to store an ionic drug in a dry state, and
at least an aqueous medium holding portion to store an aqueous medium, the drug holding portion exhibiting ion conductivity in response to a needle inserted into the aqueous medium holding portion from outside the first electrode assembly such that the drug holding portion is supplied with the aqueous medium during use and the ionic drug dissolves in the aqueous medium; and
a second electrode assembly electrically coupled to the electric power source as a counter electrode to the first electrode assembly.

23. The iontophoresis device of claim 22 wherein the first electrode assembly comprises:
a first electrode electrically coupled to the electric power source to have a same polarity as a component of the ionic drug;
a first electrolyte solution holding portion impregnated with a first electrolyte solution, the first electrolyte solution holding portion disposed adjacent to the first electrode;
a first ion exchange membrane that substantially passes ions having a polarity that is the same as a polarity of the ionic drug and that substantially blocks ions having a polarity that is opposite the polarity of the ionic drug, the ion exchange membrane disposed adjacent to the first electrolyte solution holding portion, the drug holding portion impregnated with the ionic drug and disposed adjacent to the first ion exchange membrane; and
a second ion exchange membrane that substantially passes ions having a polarity opposite the polarity of the ionic drug and that substantially blocks ions having a polarity that is the same as a polarity of the ionic drug, the second ion exchange membrane disposed adjacent to the drug holding portion.

24. The iontophoresis device of claim 23 wherein the second electrode assembly comprises:
a second electrode electrically coupled to the electric power source to have a polarity opposite that of the first electrode;
a second electrolyte solution holding portion impregnated with a second electrolyte solution, the second electrolyte solution holding portion disposed adjacent to the second electrode; and
a third ion exchange membrane that substantially passes ions having a polarity that is the same as a polarity of the ionic drug and that substantially blocks ions having a polarity opposite the polarity of the ionic drug, the third ion exchange membrane disposed adjacent to the second electrolyte solution holding portion.

25. An iontophoresis device, comprising:
an electric power source;
a first electrode assembly electrically coupled to the electric power source to store and transdermally administer an ionic drug to an organism via iontophoresis, the first electrode assembly includes:
a drug holding portion to store an ionic drug in a dry state, and
at least an aqueous medium holding portion to store an aqueous medium, the drug holding portion exhibiting ion conductivity in response to the aqueous medium holding portion being molten by heat or electricity such that the drug holding portion is supplied with the aqueous medium during use and the ionic drug dissolves in the aqueous medium; and
a second electrode assembly electrically coupled to the electric power source as a counter electrode to the first electrode assembly.

26. The iontophoresis device of claim 25 wherein the first electrode assembly comprises:
a first electrode electrically coupled to the electric power source to have a same polarity as a component of the ionic drug;
a first electrolyte solution holding portion impregnated with a first electrolyte solution, the first electrolyte solution holding portion disposed adjacent to the first electrode;
a first ion exchange membrane that substantially passes ions having a polarity that is the same as a polarity of the ionic drug and that substantially blocks ions having a polarity that is opposite the polarity of the ionic drug, the ion exchange membrane disposed adjacent to the first electrolyte solution holding portion, the drug holding portion impregnated with the ionic drug and disposed adjacent to the first ion exchange membrane; and
a second ion exchange membrane that substantially passes ions having a polarity opposite the polarity of the ionic drug and that substantially blocks ions having a polarity that is the same as a polarity of the ionic drug, the second ion exchange membrane disposed adjacent to the drug holding portion.

27. The iontophoresis device of claim 26 wherein the second electrode assembly comprises:
a second electrode electrically coupled to the electric power source to have a polarity opposite that of the first electrode;
a second electrolyte solution holding portion impregnated with a second electrolyte solution, the second electrolyte solution holding portion disposed adjacent to the second electrode; and
a third ion exchange membrane that substantially passes ions having a polarity that is the same as a polarity of the ionic drug and that substantially blocks ions having a polarity opposite the polarity of the ionic drug, the third ion exchange membrane disposed adjacent to the second electrolyte solution holding portion.

28. The iontophoresis device of claim 27 wherein the aqueous medium holding portion is molten upon energization of the iontophoresis device.
